(19) United States
(12) Patent Application Publication
(10) Pub. No.: US 2026/0325157 A1
(43) Pub. Date: Oct. 22, 2026

(54) SURGICAL DEVICE WITH RFID CHIP AND RFID ANTENNA

(71) Applicant: Aesculap AG, Tuttlingen (DE)

(72) Inventors: Andre Buerk, Villingen-Schwenningen (DE); Roland-Alois Hoegerle, Tuttlingen (DE); Frederick Lenzenhuber, Tuttlingen (DE); Thomas-Erwin Kahler, Seitingen-Oberflacht (DE); Martin Machill, Rietheim-Weilheim (DE); Simon Bohner, Villingen-Schwenningen (DE)

(21) Appl. No.: 19/117,491

(22) PCT Filed: Jul. 23, 2024
(86) PCT No.: PCT/EP2024/070844

§ 371 Date: Apr. 1, 2025

(30) Foreign Application Priority Data
Jul. 26, 2023 (DE) 10 2023 119 808.4

(51) Int. Cl. A61B 17/32; A61B 17/00

(52) U.S. Cl. CPC A61B 17/32; A61B 2017/00039; A61B 2017/00398; A61B 2017/00477

(57) ABSTRACT

A surgical device has an application part in which an RFID chip and an RFID antenna, adjacent to the RFID chip, are arranged, and has a supply cable that can be coupled to the application part. The application part and the supply cable can be coupled to one other in an OFF state, in which first coupling contacts of a first and a second coupling device are electrically in engagement and second coupling contacts are electrically out of engagement, or in an ON state, in which the first coupling contacts and the second coupling contacts are electrically in engagement. The first and the second coupling device each comprise an additional coupling contact designed such that the first and the second coupling device are electrically in engagement at least in the OFF state to supply the RFID antenna with energy.

### CROSS-REFERENCE TO RELATED APPLICATIONS

This application is the United States national stage entry of International Application No. PCT/EP2024/070844, filed on Jul. 23, 2024, and claims priority to German Application No. 10 2023 119 808.4, filed on Jul. 26, 2023. The contents of International Application No.

PCT/EP2024/070844 and German Application No. 10 2023 119 808.4 are incorporated by reference herein in their entireties.

### FIELD

The present disclosure relates to a surgical device having a surgical application part which includes an electric motor with motor windings, and an electrical supply cable which includes a number of lines corresponding to a number of the motor windings and can be coupled to the application part to supply energy to the electric motor.

### BACKGROUND

A surgical device of this type is known from DE 10 2011 050 192 A1. There is described a surgical coupling system having two coupling devices which include coupling contacts that can be mechanically and electrically in engagement with each other. A first coupling device is associated with a surgical application part and a second coupling device is associated with an electrical supply cable. The first and second coupling devices are designed so that they can assume two switching states, i.e. an OFF state and an ON state. In the OFF state, it can be determined by means of a coding/detection resistor which type of application part is coupled to the electrical supply cable. In the ON state, the electric motor in the application part then can be operated and/or activated based on this information by a control unit connected to the other end of the supply cable. It is a drawback in this case that the number of applicable resistors is limited so that only a general type of the application part, such as a milling cutter, saw, etc., can be determined. Consequently, no further data and/or information can be retrieved by the control unit.

For this reason, WO 2021/069 662 A1 discloses a surgical device having an integrated RFID readout antenna as a development of the foregoing surgical device.

### SUMMARY

It is the object of the present disclosure to provide a developed surgical device.

Advantageous embodiments are disclosed in the description and/or in the figures.

A surgical device according to the disclosure includes a surgical application part that has an electric motor with motor windings and a first coupling device at one end thereof. The electric motor can be designed as an alternating current motor or, more preferred, as a three-phase motor having three or a multiple of three motor windings.

In order to supply energy to the application part, i.e. to the electric motor, the surgical device includes an electrical supply cable with a number of lines corresponding to a number of the motor windings. As a consequence, the electrical supply cable can have two lines when using the alternating current motor. When using the three-phase motor, the electrical supply cable can have three lines to energize the three or a multiple of three, such as six, motor windings. The electrical supply cable at one end includes a second coupling device which is arranged for coupling to the first coupling device. At the second end, the electrical supply cable is connected or connectable, e.g., by means of an intended control unit coupling device, to a control unit for controlling the electric motor.

The first and second coupling devices are arranged so that they can be brought into a disconnected state or into an OFF state or into an ON state. In the disconnected state, the first and second coupling devices are completely out of engagement both mechanically and electrically. In the OFF state, the first and second coupling devices are at least partly mechanically in engagement, and at least several of the coupling contacts are electrically and mechanically in engagement. In the ON state, the first and second coupling devices are completely mechanically in engagement, and coupling contacts which are used to energize the electric motor are in engagement both electrically and mechanically. This type of the first and second coupling devices is known already from DE 10 2011 050 192 A1 the content of disclosure of which is incorporated by reference herein.

According to the present disclosure, in the OFF state, first coupling contacts of the first and second coupling devices are electrically in engagement. The first coupling contact in the first coupling device is connected to a first motor winding, and the coupling contact in the second coupling device is connected to a first line in the electrical supply cable. Further, second coupling contacts of the first and second coupling devices are electrically out of engagement. The second coupling contact in the first coupling device is connected to a second motor winding, and the second coupling contact of the second coupling device is connected to a second line in the electrical supply cable. Accordingly, in the OFF state, a supply of energy between the second line and the second motor winding is interrupted so that the electric motor is not in operation.

According to the present disclosure, in the ON state, the first coupling contacts of the first and second coupling devices and the second coupling contacts of the first and second coupling devices are electrically and mechanically in engagement. Consequently, in the ON state, the second motor winding is supplied with energy via the second line to drive the electric motor.

The surgical device includes an RFID chip disposed in the first coupling device. The RFID chip can preferably be in the form of a passive glass tag. A plurality of data and, resp., information can be stored in the RFID chip. For example, the data can be a serial number, a maintenance interval, a type of the maintenance performed, a manufacturing date, a service, an application counter and/or an application period. Accordingly, high transparency is possible in the event of service and/or fault.

In order to read out the data stored in the RFID chip, the surgical device includes an RFID antenna arranged in the first coupling device and adapted to be energized. The RFID antenna is adjacent, i.e. in direct proximity, to the RFID chip and is designed to excite the RFID chip so as to transmit data preferably bidirectionally between the RFID chip and the control unit. Consequently, the data stored in the RFID chip can be read by the control unit and the control unit can only release the use of the application part, for example, if the stored data satisfy predetermined conditions. In this way, use of an unmaintained application part can be reliably prevented, for example. Preferably, the control unit can also write data into the RFID chip so that the data are updated after use of the application part. The RFID antenna can be designed, for example, as a wire, specifically a copper wire, a printed circuit board with conductor paths, a flexible printed circuit board with conductor paths, synthetic material with activated conductor paths, as a two-component injection molding with conductor paths or as a slot antenna.

According to the disclosure, each of the first and second coupling devices includes a further electric coupling contact. The further coupling contacts of the first and second coupling devices are arranged so that they are electrically in engagement at least in the OFF state to supply the RFID antenna with energy. Accordingly, the RFID chip can be read out/written in the OFF state. Thus, no bush having a contact bridge and a resilient element for resetting the contact bridge, as it is used in WO 2021/069 662 A1, is required.

Preferably, each of the first and second coupling devices may include third coupling contacts which are electrically in engagement in the OFF state and in the ON state. As a consequence, the first and the third motor winding of the electric motor, specifically the series resistance thereof, can be measured by the control unit for determining a type of electric motor in the application part. This can be done simultaneously with receiving/writing data from/into the RFID chip.

As an alternative, the first and second coupling devices can include third coupling contacts which are electrically out of engagement in the OFF state and are electrically in engagement in the ON state. That is, the third coupling contacts are electrically in engagement at least in the ON state to allow the electric motor to be supplied with energy.

In particular, the further coupling contact in the first coupling device can be connected in parallel to the second coupling contact via the RFID antenna.

As an alternative, the further coupling contact in the first coupling device can be connected in parallel to the first coupling contact via the RFID antenna.

Advantageously, the further coupling contact and the second coupling contact or the first coupling contact can be connected to each other in parallel in the first coupling device via a wire jumper. Accordingly, a simple connection between the respective coupling contacts can be obtained. The wire jumper can also be retrofitted for existing four-pin first coupling devices, where necessary.

In addition, the further coupling contact in the second coupling device can be connected in parallel to the second coupling contact. As a consequence, in the OFF state in which the further coupling contacts are electrically in engagement, the RFID antennas can be supplied with energy via a second line connected to the second coupling contact in the electrical supply cable.

As an alternative, the further coupling contact in the second coupling device can be connected to a shield of the electrical supply cable. Accordingly, the RFID antenna can be supplied with electrical energy via the shield. Preferably, the shield should not contact the housing of the application part. Moreover, a creepage distance of at least 4 mm as well as a clearance distance having a height factor of 3.7 mm should be observed. In this way, carry-over of external voltages can be counteracted.

According to one aspect, the further coupling contacts may be designed so that they are electrically in engagement even in the ON state. In this way, it is easy to form the further coupling contacts. One of the further coupling contacts, preferably the further coupling contact in the first coupling device, can be connected in series to a detection resistor. This configuration is advantageous when each of the further coupling contacts is connected in parallel to the second coupling contacts and the second coupling contacts in the OFF state are bridged by the further coupling contacts. The detection resistor preferably can be arranged in the application part and can be encoded corresponding to the application part. Accordingly, the application part can be identified by measuring the detection resistor. Moreover, in the ON state current is not conducted via the further coupling contacts but via the second coupling contacts.

Alternatively, the further coupling contacts can be designed so that they are electrically out of engagement in the ON state. Consequently, a current flow via the further coupling contacts is reliably suppressed in the ON state.

Even when using the further coupling contacts which are electrically out of engagement in the ON state, one of the further coupling contacts, preferably the further coupling contact in the first coupling device, can be connected in series to a detection resistor.

As a consequence, the detection resistor can be arranged in the first coupling device or in the second coupling device. Preferably, the detection resistor can be arranged in the first coupling device and can have a resistance value corresponding to the application part or the electric motor. The detection resistor can have a resistance value of 10 ohms, for example. Accordingly, a series resistance between the detection resistor and the resistance of the RFID antenna can be detected before and/or during excitation of the RFID antenna by the control unit in order to determine that the OFF state is correctly provided. This procedure is primarily advantageous when the third coupling contacts of the first coupling device and the second coupling device in the OFF state are electrically out of engagement and no measurement of the series resistance of motor windings is possible. Since the series resistance of the motor windings is in the range of few ohms, it is also advantageous, however, to provide the detection resistor even when the series resistance of the motor windings can be measured via the third coupling contacts.

Furthermore, the coupling contacts can be in the form of pins in the first coupling device and in the form of bushes in the second coupling device. The pin of the further coupling contact can be longer than the pin of the second coupling contact. Consequently, an electrically conducting contact of the further coupling contacts is obtained in the OFF state while the second coupling contacts are out of engagement electrically as well as mechanically. The pin of the further coupling contact may include an insulating portion that is designed so that the further coupling contacts in the ON state are electrically out of engagement. In particular, a shank portion of the coupling contact in the form of a pin can be covered or coated with an insulating layer to form the insulating portion. Preferably, the insulating layer is formed so that it is substantially level with a conductive portion at the tip of the pin. Alternatively, or additionally, the bush of the further coupling contact may include an insulating portion that is designed so that the third coupling contacts are electrically out of engagement in the ON state. Particularly, a bottom portion of the bush can be covered by an insulating layer to form the insulating portion. Preferably, the insulating layer is formed to be substantially level with a conductive portion on an inlet portion of the bush. In this way, an electrically conductive connection of the further coupling contacts is obtained in the OFF state and an electrically non-conductive connection is obtained in the ON state.

A surgical device according to the disclosure comprises a surgical application part which has an electric motor with motor windings and a first coupling device at one end. The electric motor can be an alternative current motor or, more preferred, a three-phase motor with three or a multiple of three motor windings.

In order to supply energy to the application part, i.e. the electric motor, the surgical device includes an electrical supply cable having a number of lines corresponding to a number of the motor windings. Consequently, the electrical supply cable can have two lines when using the alternating current motor. When using the three-phase motor, the electrical supply cable can have three lines to supply the three or a multiple of three, such as six, motor windings with energy. The electrical supply cable at one end includes a second coupling device that is arranged for coupling to the first coupling device. At the second end, the electrical supply cable is connected or connectable, e.g., by means of an intended control unit coupling device, to a control unit for controlling the electric motor.

The first and second coupling devices are arranged so that they can be brought into a disconnected state or into an OFF state or into an ON state. In the disconnected state, the first and second coupling devices are completely out of engagement both mechanically and electrically. In the OFF state, the first and second coupling devices are at least partly mechanically in engagement, and at least several of the coupling contacts are electrically and mechanically in engagement. In the ON state, the first and second coupling devices are completely mechanically in engagement, and coupling contacts that serve for supplying the electric motor with energy are in engagement both electrically and mechanically. This type of the first and the second coupling device is known already from DE 10 2011 050 192 A1 the content of disclosure of which is incorporated by reference herein.

According to the present disclosure, in the OFF state first coupling contacts of the first and second coupling devices are electrically in engagement. The first coupling contact in the first coupling device is connected to a first motor winding, and the first coupling contact in the second coupling device is connected to a first line in the electrical supply cable. Further, second coupling contacts of the first and second coupling devices are electrically out of engagement. The second coupling contact in the first coupling device is connected to a second motor winding, and the second coupling contact of the second coupling device is connected to a second line in the electrical supply cable. Accordingly, in the OFF state a supply of energy between the second line and the second motor winding is interrupted so that the electric motor is not in operation.

According to the present disclosure, in the ON state the first coupling contacts of the first and second coupling devices and the second coupling contacts of the first and second coupling devices are electrically and mechanically in engagement. Consequently, in the ON state, energy is supplied to the second motor winding via the second line to drive the electric motor.

The surgical device includes an RFID chip disposed in the first coupling device. Preferably, the RFID chip can be designed as a passive glass tag. A plurality of data and, resp., information can be stored in the RFID chip. The data can comprise, for example, a serial number, a maintenance interval, a type of the maintenance work carried out, a manufacturing date, a performance, an application counter and/or an application period. Consequently, a high level of transparency is made possible in the event of service or fault.

In order to read out the data stored in the RFID chip, the surgical device includes an RFID antenna arranged in the second coupling device and adapted to be supplied with energy. The RFID antenna is adjacent, i.e. in direct proximity, to the RFID chip at least in the OFF state and is designed to excite the RFID chip to transmit data preferably bidirectionally between the RFID chip and the control unit. Therefore, the data stored in the RFID chip can be read by the control unit, and use of the application part can only be released by the control unit, for example, when the stored data satisfy predetermined conditions. In this way, for example use of an unmaintained application part can be reliably prevented. Preferably, the control unit also can write data into the RFID chip so that the data are updated after use of the application part. The RFID antenna can be in the form of a wire, specifically copper wire, printed circuit board with conductor paths, flexible printed circuit board with conductor paths, synthetic material with activated conductor paths, as a two-component injection molding with conductor paths or as a slot antenna.

According to the disclosure, each of the first and second coupling devices has a further electric coupling contact. The further coupling contacts of the first and second coupling devices are arranged so that they are electrically in engagement at least in the OFF state in order to supply the RFID antenna with energy. Therefore, the RFID chip can be read out/written in the OFF state. As a result, no bush with a contact bridge and a resilient element to reset the contact bridge, as it is used in WO 2021/069 662 A1, is required.

Preferably, each of the first and second coupling devices can have third coupling contacts which are electrically in engagement in the OFF state and in the ON state. Consequently, the first and third motor windings of the electric motor, specifically the serial resistance thereof, can be measured by the control unit to determine a type of the electric motor in the application part. This can be done simultaneously with receiving/writing data from/into the RFID chip.

As an alternative, the first and second coupling devices can have third coupling contacts which are electrically out of engagement in the OFF state and are electrically in engagement in the ON state. That is, the third coupling contacts are electrically in engagement at least in the ON state to allow the electric motor to be supplied with energy.

In particular, the further coupling contact in the first coupling device can be connected in parallel to the second coupling contact.

As an alternative, the further coupling contact in the first coupling device can be connected in parallel to the first coupling contact.

Advantageously, the further coupling contact and the second coupling contact or the first coupling contact in the first coupling device can be connected in parallel to each other via a wire jumper. Consequently, a simple connection between the respective coupling contacts can be obtained. The wire jumper can also be retrofitted for existing four-pin first coupling devices, where necessary.

In addition, the further coupling contact in the second coupling device can be connected in parallel to the second coupling contact via the RFID antenna. As a consequence, in the OFF state in which the further coupling contacts are electrically in engagement, the RFID antennas can be supplied with energy via a second line connected to the second coupling contact in the electrical supply cable.

As an alternative, the further coupling contact in the second coupling device can be connected to a shield of the electrical supply cable via the RFID antenna. Therefore, the RFID antenna can be supplied with electrical energy via the shield. Preferably, the shield should not contact the housing of the application part. Moreover, a creepage distance of at least 4 mm as well as a clearance distance with a height factor of 3.7 mm should be observed. In this way, carry-over of external voltages can be counteracted.

In accordance with one aspect, the further coupling contacts can be configured so that they are electrically in engagement also in the ON state. In this way, it is easy to form the further coupling contacts. One of the further coupling contacts, preferably the further coupling contact in the first coupling device, may be connected in series to a detection resistor. This configuration is of advantage when each of the further coupling contacts is connected in parallel to the second coupling contacts and the second coupling contacts are bridged in the OFF state. The detection resistor can preferably be arranged in the application part and can be encoded corresponding to the application part. Consequently, the application part can be identified by measuring the detection resistor. Moreover, in the ON state current is not conducted via the further coupling contacts but via the second coupling contacts.

As an alternative, the further coupling contacts can be configured so that they are electrically out of engagement in the ON state. Consequently, a current flow is reliably suppressed in the ON state via the further coupling contacts.

Even when using the further coupling contacts which are electrically out of engagement in the ON state, one of the further coupling contacts, preferably the further coupling contact in the first coupling device, can be connected in series to a detection resistor.

The detection resistor consequently can be arranged in the first coupling device or in the second coupling device. Preferably, the detection resistor can be arranged in the first coupling device and can have a resistance value corresponding to the application part or to the electric motor. The detection resistor may have a resistance value of 10 ohms, for example. Therefore, a series resistance between the detection resistor and the resistance of the RFID antenna can be detected by the control unit before and/or during excitation of the RFID antenna to determine the correct presence of the OFF state. This procedure is mainly advantageous when the third coupling contacts of the first coupling device and the second coupling device in the OFF state are electrically out of engagement and measurement of the series resistance of motor windings is not possible. Since the series resistance of the motor windings is in the range of few ohms, it is also advantageous to provide the detection resistor, however, when the series resistance of the motor windings can be measured via the third coupling contacts.

In addition, the coupling contacts can be in the form of pins in the first coupling device and in the form of bushes in the second coupling device. The pin of the further coupling contact can be longer than the pin of the second coupling contact. Consequently, an electrically conducting contact of the further coupling contacts is achieved in the OFF state, while the second coupling contacts are electrically as well as mechanically out of engagement. The pin of the further coupling contact can include an insulating portion which is designed so that the further coupling contacts are electrically out of engagement in the ON state. In particular, a shank portion of the coupling contact in the form of a pin can be covered or coated with an insulating layer to form the insulating portion. The insulating layer preferably is formed so that it is substantially level with a conductive portion at the tip of the pin. Alternatively, or additionally, the bush of the further coupling contact can include an insulating portion which is configured so that the third coupling contacts are electrically out of engagement in the ON state. Particularly, a bottom portion of the bush may be covered by an insulating layer to form the insulating portion. The insulating layer is preferably formed substantially level with a conductive portion on an inlet portion of the bush. In this way, an electrically conductive connection of the further coupling contacts is achieved in the OFF state and an electrically non-conductive connection is achieved in the ON state.

A surgical device according to the disclosure comprises a surgical application part which includes an electric motor with motor windings and a first coupling device at one end. The electric motor can be an alternating current motor or, more preferably, a three-phase motor with three or a multiple of three motor windings.

In order to supply energy to the application part, i.e. the electric motor, the surgical device includes an electrical supply cable with a number of lines corresponding to a number of the motor windings. Consequently, the electrical supply cable can include two lines when using the alternating current motor. When using the three-phase motor, the electrical supply cable can include three lines to supply energy to the three or a multiple of three, such as six, motor windings. The electrical supply cable at one end includes a second coupling device that is arranged for coupling to the first coupling device. At the second end, the electrical supply cable is connected or connectable, e.g. by means of an intended control unit coupling device, to a control unit for controlling the electric motor.

The first and second coupling devices are arranged so that they can be brought into a disconnected state or into an OFF state or into an ON state. In the disconnected state, the first and second coupling devices are completely out of engagement both mechanically and electrically. In the OFF state, the first and second coupling devices are at least partly mechanically in engagement and at least several of the coupling contacts are electrically and mechanically in engagement. In the ON state, the first and second coupling devices are completely mechanically in engagement, and coupling contacts that serve for supplying the electric motor with energy are in engagement both electrically and mechanically. This type of the first and second coupling devices is known already from DE 10 2011 050 192 A1 the content of disclosure of which is incorporated by reference herein.

According to the present disclosure, in the OFF state first coupling contacts of the first and second coupling devices are electrically in engagement. The first coupling contact in the first coupling device is connected to a first motor winding, and the first coupling contact in the second coupling device is connected to a first line in the electrical supply cable. Furthermore, second coupling contacts of the first and second coupling devices are electrically out of engagement. The second coupling contact in the first coupling device is connected to a second motor winding, and the second coupling contact of the second coupling device is connected to a second line in the electrical supply cable. Consequently, in the OFF state an energy supply between the second line and the second motor winding is interrupted so that the electric motor is not in operation.

According to the present disclosure, in the ON state the first coupling contacts of the first and second coupling devices and the second coupling contacts of the first and second coupling devices are electrically and mechanically in engagement. Accordingly, in the ON state the second motor winding is supplied with energy via the second line to drive the electric motor.

The surgical device includes an RFID chip arranged in the first coupling device. Preferably, the RFID chip can be designed as a passive glass tag. A plurality of data and information can be stored in the RFID chip. The data may comprise, for example, a serial number, a maintenance interval, a type of the maintenance work carried out, a manufacturing date, a performance, an application counter and/or an application period. Consequently, a high level of transparency is made possible in the event of service or fault.

In order to read out the data stored in the RFID chip, the surgical device includes an RFID antenna arranged in the second coupling device and adapted to be energized. The RFID antenna is arranged at least in the OFF state adjacent, i.e. in direct proximity, to the RFID chip and is configured to excite the RFID chip so as to transmit data preferably bidirectionally between the RFID chip and the control unit. Preferably, the RFID antenna is arranged adjacent to the RFID chip also in the ON state. Accordingly, the data stored in the RFID chip can be read by the control unit and use of the application part can be released by the control unit only when the stored data satisfy predetermined conditions, for example. In this way, use of an unmaintained application part can be reliably avoided, for example. Of preference, the control unit can also write data into the RFID chip so that the data are updated after use of the application part. The RFID antenna can be in the form of a wire, specifically copper wire, printed circuit board with conductor paths, flexible printed circuit board with conductor paths, synthetic material with activated conductor paths, as two-component injection mold with conductor paths or as a slot antenna.

Moreover, two further lines are provided in the electrical supply cable. The two further lines are connected to the RFID antenna in the second coupling device to energize the RFID antenna. The further lines may have a smaller cross-section than the lines provided for energizing the electric motor. In addition, the two lines are directly connected to the RFID antenna. In this way, a reliable energy supply of the RFID antenna is achieved both in the OFF state and in the ON state.

One of the further lines can be provided by an umbrella of the supply cable. Accordingly, only one further line has to be provided in the electrical supply cable in addition to the lines serving for energizing the electric motor.

As an alternative, the further lines can be provided by a coax cable. Accordingly, a propagation of electromagnetic disturbances which occur when the RFID antenna is energized is suppressed.

Further, each of the first and second coupling devices can include a further coupling contact. Each of the further coupling contacts can be connected in parallel to the second coupling contact and can be arranged so that they are electrically in engagement at least in the OFF state. One of the further coupling contacts, preferably the further coupling contact in the first coupling device, can be connected in series to a detection resistor. The detection resistor can preferably be arranged in the application part and can be encoded corresponding to the application part. Consequently, the application part can be identified by measuring the detection resistor. Moreover, in the ON state current is not conducted via the further coupling contacts but via the second coupling contacts.

A surgical device according to the disclosure comprises a surgical application part that includes an electric motor with motor windings and a first coupling device at one end. The electric motor can be in the form of an alternating current motor or, more preferred, of a three-phase motor with three or a multiple of three motor windings.

In order to supply energy to the application part, i.e. the electric motor, the surgical device includes an electrical supply cable having a number of lines corresponding to a number of the motor windings. As a consequence, the electrical supply cable can have two lines when the alternating current motor is used. When using the three-phase motor, the electrical supply cable can have three lines to energize the three or the multiple of three, such as six, motor windings. At one end, the electrical supply cable includes a second coupling device that is arranged for coupling to the first coupling device. At the second end, the electrical supply cable is connected or connectable, e.g. by means of an intended control unit coupling device, to a control unit for controlling the electric motor.

The first and second coupling devices are arranged so that they can be brought into a disconnected state or into an OFF state or into an ON state. In the disconnected state, the first and second coupling devices are completely out of engagement both mechanically and electrically. In the OFF state, the first and second coupling devices are at least partially mechanically in engagement, and at least several of the coupling contacts are electrically and mechanically in engagement. In the ON state, the first and second coupling devices are completely mechanically in engagement, and coupling contacts that serve to energize the electric motor are in engagement both electrically and mechanically. This type of the first and second coupling devices is known already from DE 10 2011 050 192 A1 the content of disclosure of which is included here by reference.

According to the present disclosure, in the OFF state, first coupling contacts of the first and second coupling devices are electrically in engagement. The first coupling contact in the first coupling device is connected to a first motor winding and the first coupling contact in the second coupling device is connected to a first line in the electrical supply cable. Furthermore, second coupling contacts of the first and second coupling devices are electrically out of engagement. The second coupling contact in the first coupling device is connected to a second motor winding, and the second coupling contact of the second coupling device is connected to a second line in the electrical supply cable. Consequently, in the OFF state, energy supply between the second line and the second motor winding is interrupted so that the electric motor is out of operation.

According to the present disclosure, in the ON state, the first coupling contacts of the first and second coupling devices and the second coupling contacts of the first and second coupling devices are electrically and mechanically in engagement. Accordingly, in the ON state, the second motor winding is energized via the second line to drive the electric motor.

The surgical device comprises an RFID chip disposed in the first coupling device. Preferably, the RFID chip can be designed as a passive glass tag. A plurality of data and information can be stored in the RFID chip. The data can comprise, for example, a serial number, a maintenance interval, a type of maintenance work carried out, a manufacturing date, a performance, an application counter and/or an application period. Consequently, a high level of transparency is made possible in the event of service and/or fault.

In order to read out the data stored in the RFID chip, the surgical device comprises an RFID antenna arranged in the first coupling device and adapted to be supplied with energy. The RFID antenna is arranged adjacent, i.e. in direct proximity, to the RFID chip and is configured to excite the RFID chip in order to transmit data preferably bidirectionally between the RFID chip and the control unit. Preferably, the RFID antenna is arranged adjacent to the RFID chip also in the ON state. Consequently, the data stored in the RFID chip can be read by the control unit, and use of the application part can be released by the control unit, for example, only when the stored data satisfy predetermined conditions. In this way, for example the use of an unmaintained application part can be reliably prevented. Preferably, the control unit can also write data into the RFID chip so that the data are updated after using the application part. The RFID antenna can be in the form of a wire, specifically copper wire, printed circuit board with conductor paths, flexible printed circuit board with conductor paths, synthetic material with activated conductor paths, as a two-component injection molding with conductor paths or as a slot antenna, for example.

Moreover, two further lines are provided in the electrical supply cable. The two further lines are connected to the RFID antenna in the second coupling device in order to supply the RFID antenna with energy. The further lines can have a smaller cross-section than the lines provided to energize the electric motor. In addition, the two lines are connected directly to the RFID antenna. In this way, reliable supply of energy to the RFID antenna is achieved in the OFF state as well as in the ON state.

One of the further lines can be provided by a shield of the supply cable. Consequently, only one further line has to be provided in the electrical supply cable in addition to the lines that are used to supply the electric motor with energy.

Alternatively, the further lines can be provided by a coax cable. Therefore, propagation of electromagnetic interference occurring when the RFID antenna is supplied with energy is suppressed.

Further, each of the first and second coupling devices can include a further coupling contact. The further coupling contacts can be connected each in parallel to the second coupling contact and can be arranged so that they are electrically in engagement at least in the OFF state. One of the further coupling contacts, preferably the further coupling contact in the first coupling device, can be connected in series to a detection resistor. The detection resistor preferably can be arranged in the application part and can be encoded corresponding to the application part. As a consequence, the application part can be identified by measuring the detection resistor. Moreover, in the ON state current is not conducted via the further coupling contacts but via the second coupling contacts.

A surgical device according to the disclosure comprises a surgical application part that includes an electric motor with motor windings and a first coupling device at one end. The electric motor can be designed as an alternating current motor or, more preferred, as a three-phase motor with three or a multiple of three motor windings.

In order to supply energy to the application part, i.e. the electric motor, the surgical device includes an electrical supply cable with a number of lines corresponding to a number of the motor windings. Consequently, the electrical supply cable can have two lines when using the alternating current motor. When using the three-phase motor, the electrical supply cable can have three lines to supply the three or the multiple of three, such as six, motor windings with energy. The electrical supply cable at one end includes a second coupling device arranged for coupling to the first coupling device. At the second end, the electrical supply cable is connected or connectable, e.g. by means of an intended control unit coupling device, to a control unit for controlling the electric motor.

The first and second coupling devices are arranged so that they can be brought into a disconnected state or into an OFF state or into an ON state. In the disconnected state, the first and second coupling devices are completely out of engagement both mechanically and electrically. In the OFF state, the first and second coupling devices are at least partly mechanically in engagement, and at least several of the coupling contacts are electrically and mechanically in engagement. In the ON state, the first and second coupling devices are completely mechanically in engagement, and coupling contacts which are used to supply the electric motor with energy are in engagement both electrically and mechanically. This type of the first and second coupling devices is known already from DE 10 2011 050 192 A1 the content of disclosure of which is incorporated by reference herein.

According to the present disclosure, first coupling contacts of the first and second coupling devices are electrically in engagement in the OFF state. The first coupling contact in the first coupling device is connected to a first motor winding, and the first coupling contact in the second coupling device is connected to a first line in the electrical supply cable. Furthermore, second coupling contacts of the first and second coupling devices are electrically out of engagement. The second coupling contact in the first coupling device is connected to a second motor winding, and the second coupling contact of the second coupling device is connected to a second line in the electrical supply cable. As a result, energy supply between the second line and the second motor winding is interrupted in the OFF state so that the electric motor is not in operation.

According to the present disclosure, the first coupling contacts of the first and second coupling devices and the second coupling contacts of the first and second coupling devices are electrically and mechanically in engagement in the ON state. Consequently, the second motor winding is supplied with energy via the second line in the ON state to drive the electric motor.

The surgical device comprises an RFID chip disposed in the first coupling device. The RFID chip preferably can be in the form of a passive glass tag. A plurality of data and information can be stored in the RFID chip. The data can comprise, for example, a serial number, a maintenance interval, a type of maintenance work carried out, a manufacturing date, a performance, an application counter and/or an application period. As a result, a high level of transparency is made possible in the event of service or fault.

In order to read out the data stored in the RFID chip, the surgical device comprises an RFID antenna arranged in the first coupling device and adapted to be supplied with energy. The RFID antenna is adjacent, i.e. in direct proximity, to the RFID chip and configured to excite the RFID chip to transmit data preferably bidirectionally between the RFID chip and the control unit. Therefore, the data stored in the RFID chip can be read by the control unit, and use of the application part can be released by the control unit only when the stored data satisfy predetermined conditions, for example. In this way, for example use of an unmaintained application part can be reliably prevented. Preferably, the control unit can also write data into the RFID chip so that the data are updated after use of the application part. The RFID antenna can be designed as a wire, specifically copper wire, printed circuit board with conductor paths, flexible printed circuit board with conductor paths, synthetic material with activated conductor paths, as a two-component injection molding with conductor paths or as a slot antenna.

Each of the first and second coupling devices has two RFID coupling contacts which are electrically in engagement at least in the OFF state. Preferably, the two RFID coupling contacts of the first and second coupling devices are electrically in engagement also in the ON state, however. The two RFID coupling contacts are connected to the RFID antenna in the first coupling device. That is, the RFID antenna in the first coupling device is connected between the two RFID coupling contacts. The two RFID coupling contacts in the second coupling device are connected to two further lines which are provided in the electrical supply cable for supplying the RFID antenna with energy. Accordingly, the RFID antenna can be reliably supplied with energy. In case that the RFID coupling contacts are electrically in engagement also in the ON state, the RFID chip can be read out by the RFID antenna even during operation of the application part.

One of the further lines can be provided by a shield of the supply cable. Consequently, only one further line has to be provided in the electrical supply cable in addition to the lines which serve for supplying the electric motor with energy.

As an alternative, the further lines can be provided by a coax cable. Accordingly, propagation of electromagnetic interference occurring when supplying the RFID antenna with energy is suppressed.

Moreover, each of the first and second coupling devices can include a further coupling contact. The further coupling contacts can be connected each in parallel to the second coupling contact and can be arranged so that they are electrically in engagement at least in the OFF state. One of the further coupling contacts, preferably the further coupling contact in the first coupling device, can be connected in series to a detection resistor. The detection resistor can preferably be arranged in the application part and can be encoded corresponding to the application part. Consequently, the application part can be identified by measuring the detection resistor. Moreover, in the ON state current is not conducted via the further coupling contacts but via the second coupling contacts.

### BRIEF DESCRIPTION OF THE DRAWINGS

In the following, the present disclosure will be described with reference to the Figures, wherein:

FIG. 1 shows a surgical device comprising a surgical application part and an electrical supply cable which are coupled to each other in an OFF state;

FIG. 2 shows the surgical device comprising the surgical application part and the electrical supply cable which are coupled to each other in an ON state;

FIG. 3 shows the surgical application part;

FIG. 4 shows a first coupling device of the surgical application part;

FIG. 5 shows a sectional view of the surgical application part;

FIG. 6 shows the electrical supply cable;

FIG. 7 shows a coupling pin of a second coupling device of the electrical supply cable;

FIG. 8 shows a rotated view of the coupling pin of the second coupling device;

FIG. 9 shows a sectional view of the first and second coupling devices which are coupled to each other in the OFF state;

FIG. 10 shows a detailed sectional view of the first and second coupling devices which are coupled to each other in the OFF state;

FIG. 11 shows a further detailed sectional view of the first and second coupling devices which are coupled to each other in the OFF state;

FIG. 12 shows a different detailed sectional view of the first and second coupling devices which are coupled to each other in the OFF state;

FIG. 13 shows a detailed sectional view of the first and second coupling devices which are coupled to each other in the ON state;

FIG. 14 shows a further detailed sectional view of the first and second coupling devices which are coupled to each other in the ON state;

FIG. 15 shows a schematic circuit diagram in the OFF state;

FIG. 16 shows a schematic circuit diagram in the OFF state illustrating a conducting path for measuring a series resistance of two motor windings;

FIG. 17 shows a schematic circuit diagram in the OFF state illustrating a conducting path for supplying an RFID antenna with energy;

FIG. 18 shows a schematic circuit diagram in the ON state;

FIG. 19 shows a first coupling device of the surgical application part;

FIG. 20 shows a schematic circuit diagram in the OFF state illustrating a conducting path for supplying an RFID antenna with energy;

FIG. 21 shows a schematic circuit diagram in the ON state;

FIG. 22 shows a schematic circuit diagram in the OFF state;

FIG. 23 shows a schematic circuit diagram in the OFF state; and

FIG. 24 shows a schematic circuit diagram in the OFF state.

### DETAILED DESCRIPTION

Hereinafter, embodiments of the present disclosure will be described with reference to the Figures. It is noted that the same reference numeral is assigned to like or functionally corresponding parts and/or portions.

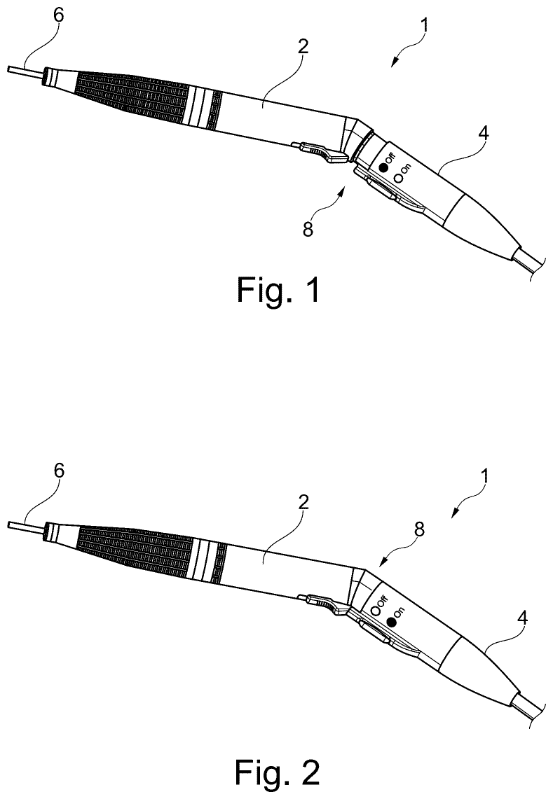

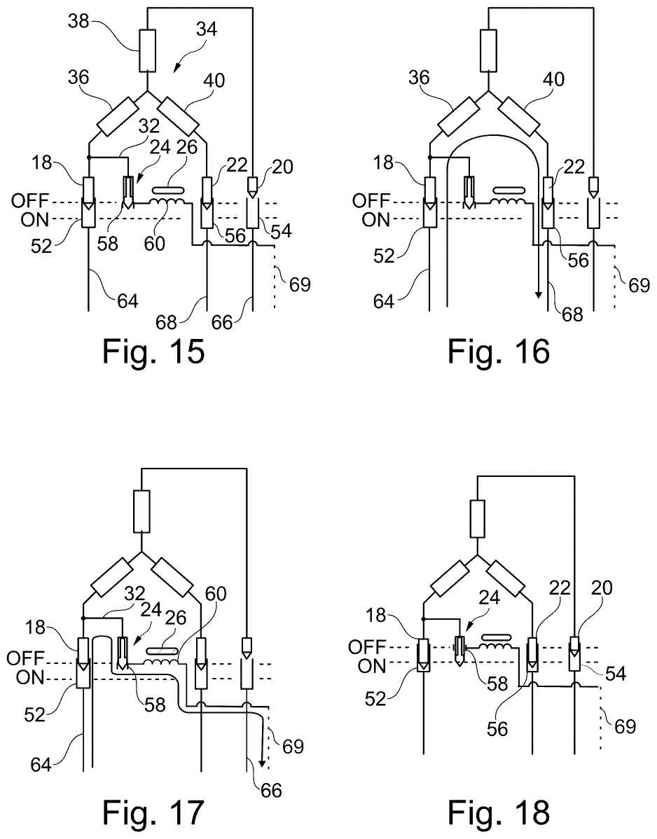

FIGS. 1 and 2 illustrate a surgical device 1 that can be a surgical application part 2 which can be, e.g., a milling cutter, a saw, etc., and an electrical supply cable 4. A tool 6 drivable by an electric motor 34 arranged in the application part 2 (see FIG. 15) is disposed at a distal end of the application part 2. The application part 2 is connected to the electrical supply cable 4 at a proximal end by a coupling system 8 (known from DE 10 2011 050 192 A1). The coupling system 8 is configured so that it enables two switching states or coupling states, that is, an OFF state (FIG. 1) and an ON state (FIG. 2).

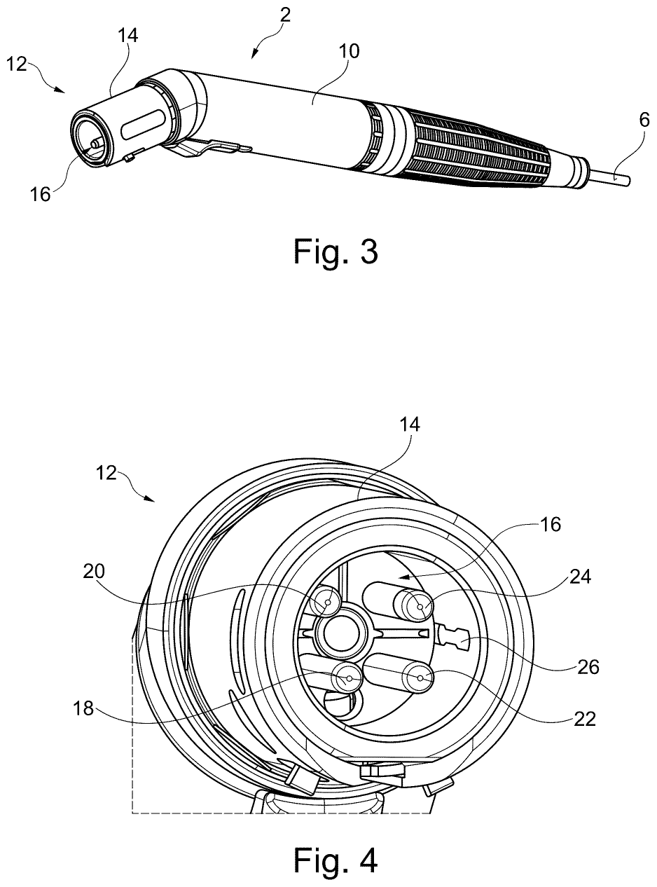

FIG. 3 illustrates the surgical application part 2 in a disconnected state, i.e. in a state in which the coupling system 8 is out of engagement. In the present case, the application part 2 is designed as an application part that can be held and/or operated manually and, therefore, includes a handle 10. A first coupling device 12 of the coupling system 8 is arranged at a proximal end of the application part 2. In the present case, the coupling device 12 is designed as a coupling pin 14 having a coupling sleeve 16 formed therein.

FIG. 4 illustrates a detailed view of the coupling device 12. In the coupling sleeve 16, four coupling contacts, viz. a first coupling contact 18, a second coupling contact 20, a third coupling contact 22 and a fourth or further coupling contact 24, are arranged which are in the form of pins in the present case. An RFID chip 26 designed a passive glass tag herein is arranged substantially level with the inner surface of the coupling sleeve 16 on an inner surface of the coupling sleeve 16 of the first coupling device 12.

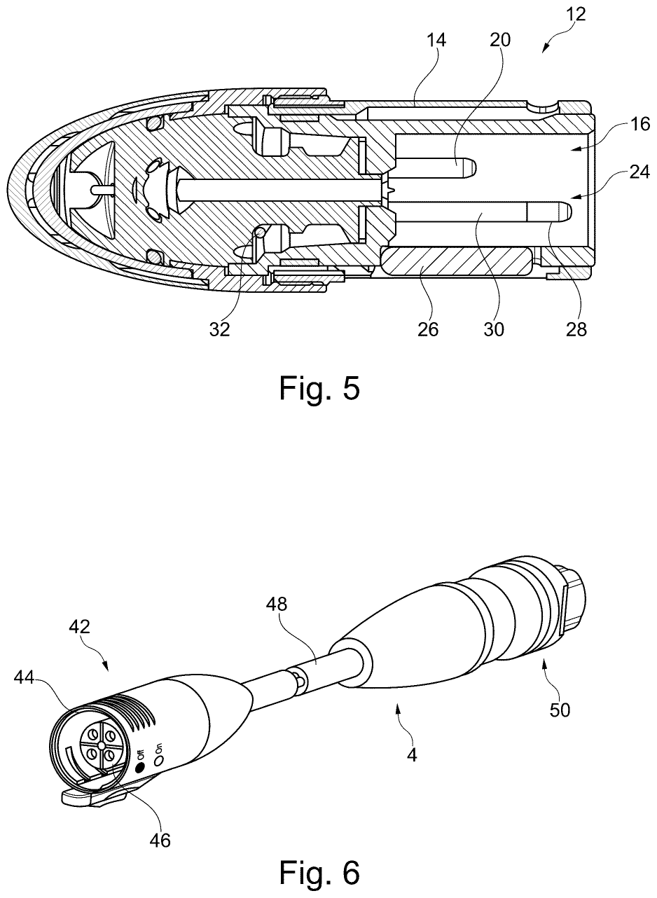

In FIG. 5, a sectional view of the first coupling device 12 can be seen in a plane of the second coupling contact 20 and the fourth coupling contact 24. It can be recognized that the second coupling contact 20 is shorter than the fourth coupling contact 24. Moreover, the fourth coupling contact 24 includes two portions, namely a conductive portion 28 at its tip and an insulating portion 30 at its shank. In addition, a wire jumper 32 can be seen.

A connection of the coupling contacts 18, 20, 22, 24 shall be illustrated by way of FIG. 15. The first coupling contact 18 is connected to a first motor winding 36. The second coupling contact 20 is connected to the second motor winding 38. The third coupling contact 22 is connected to the third motor winding 40. The fourth coupling contact 24 is connected in the application part 2 in parallel to the first coupling contact 18 via the wire jumper 32.

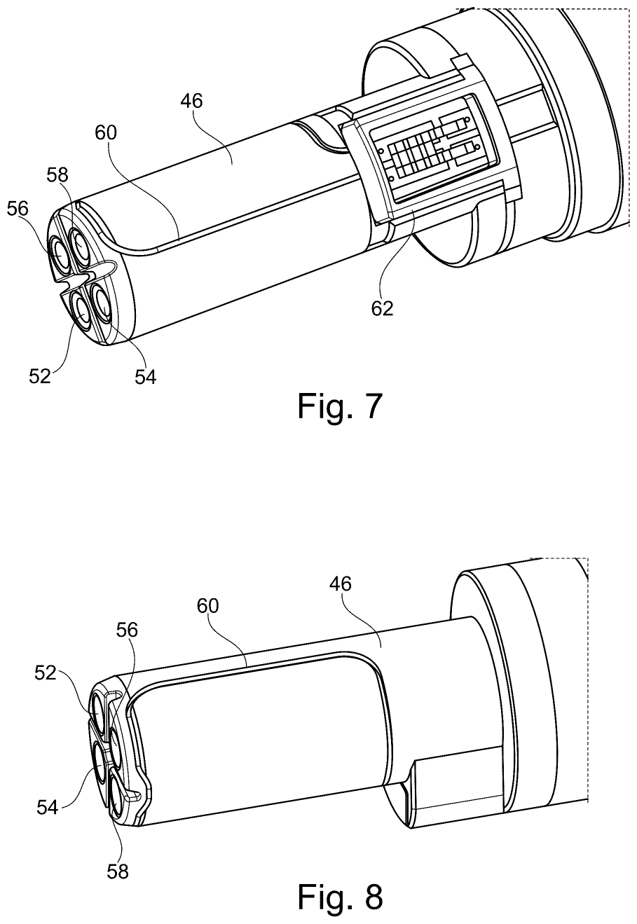

FIG. 6 illustrates the electrical supply cable 4 having, at its distal end, a second coupling device 42 which is provided as a coupling sleeve 44 including a coupling pin 46 formed therein. Thus, the second coupling device 42 is arranged to be mechanically in engagement with the first coupling device 12. The second coupling device 34 is connected, via a cable portion 48 in which a number of lines corresponding to the number of the motor windings extends, to a control unit coupling device 50 configured to be connectable to a control unit for operating the electric motor 34 FIGS. 7 and 8 illustrate a detailed view of the coupling pin 46 without the coupling sleeve 44. Four coupling contacts, namely a first coupling contact 52, a second coupling contact 54, a third coupling contact 56 and a fourth or further coupling contact 58, are embedded or inserted in the coupling pin 46. In the present case, the coupling contacts 52, 54, 56, 58 are designed as bushes which can be made to mesh mechanically with the coupling contacts 18, 20, 22, 24 in the form of pins. An RFID antenna 60 is embedded in an intended groove on an outer surface of the coupling pin 46. In the present case, the RFID antenna 60 is a copper wire loop.

The copper wire loop extends on the outer face of the coupling pin initially in the axial direction to an end face of the coupling pin 46. From there, the loop then extends about a predetermined angle, such as 180°, along the circumferential direction of the coupling pin 46, i.e., in parallel to an end face of the coupling pin 46. After that, the loop returns via a portion in the axial direction and a portion along the circumferential direction to the proximity of its starting point. Accordingly, the RFID antenna 60 can be easily arranged on the outer surface of the coupling pin 46. It is noted that this type of RFID antenna 60 is exemplary and other antenna shapes, such as meander-shaped, spiral-shaped, coil-shaped or helix-shaped, are possible. Alternatively, also a different type of antenna, such as printed circuit board with conductor paths, flexible printed circuit board with conductor paths, synthetic material with activated conductor paths, two-component injection molding with conductor paths or slot antenna, can be used.

The second coupling device 42 includes an electric circuit 62 that is connected to the RFID antenna 60. The electric circuit 62 may be configured so that it tunes the RFID antenna 60 to allow reliable data transmission between the RFID chip 26 and the control unit via the RFID antenna 60. Alternatively, or additionally, the electric circuit 62 may be configured so that it processes data received from the RFID antenna 60 and transmits the data to the control unit via line 64 and a shield 69 (see FIG. 15) of the electrical supply cable 4, e.g. using the one-wire technology, and/or processes the data received from the control unit, e.g. using the one-wire technology, and writes them into the RFID chip 26. Consequently, the electrical supply cable 4 can have a larger length without a data transmission being disturbed.

A connection of the coupling contacts 52, 54, 56, 58 is described by way of FIG. 15. The first coupling contact 52 is connected to a first line 64. The second coupling contact 54 is connected to a second line 66. The third coupling contact 56 is connected to a third line 68. As described above, the lines 64, 66, 68 extend in the cable portion 48 of the electrical supply cable 4. The fourth coupling contact 58 is connected to the second line 66 a shield 69 of the electrical supply cable in the second coupling device.

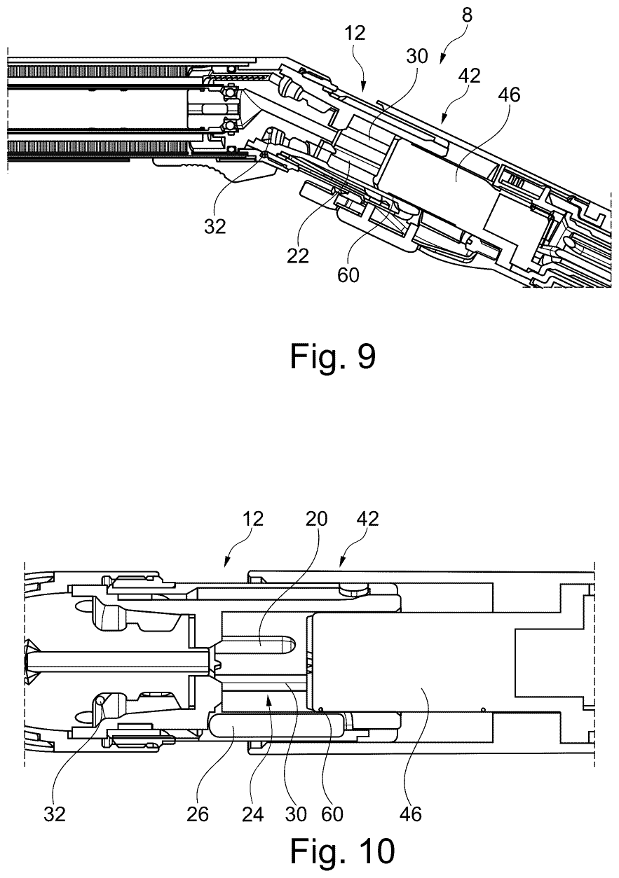

Hereinafter, switching states of the coupling system 8 shall be described. In FIG. 9, the first coupling device 12 and the second coupling device 42 are partly in engagement to reach the OFF state. It can be seen that the third coupling contact 22 and the fourth coupling contact 24, i.e. the conductive portion 28, partially protrude into the intended coupling contacts 56 and, resp., 58 in the coupling pin 46. As a consequence, the insulating portion 30 is arranged outside the fourth coupling contact 58. In addition, the wire jumper 32 connecting the first coupling contact 18 to the fourth coupling contact 24 can be recognized. In the view shown in FIG. 9, the antenna 60 is arranged so that the loop portion is positioned along the circumferential direction behind the image plane in order to be adjacent to the RFID chip 26.

FIG. 10 also illustrates a case in which the coupling system 8 is in the OFF state. It can be seen that the second coupling contact 20 is shorter than the other coupling contacts 18, 22, 24 so that, in the OFF state, is does not protrude into the intended second coupling contact 54 in the coupling pin 46. Accordingly, a connection between the second line 66 and the second motor windings 38 is interrupted to stop the operation of the electric motor 34. Moreover, FIG. 10 shows the arrangement of the RFID antenna 60, particularly the portion extending along the circumferential direction of the coupling pin 46, adjacent to the RFID chip 26 in the OFF state.

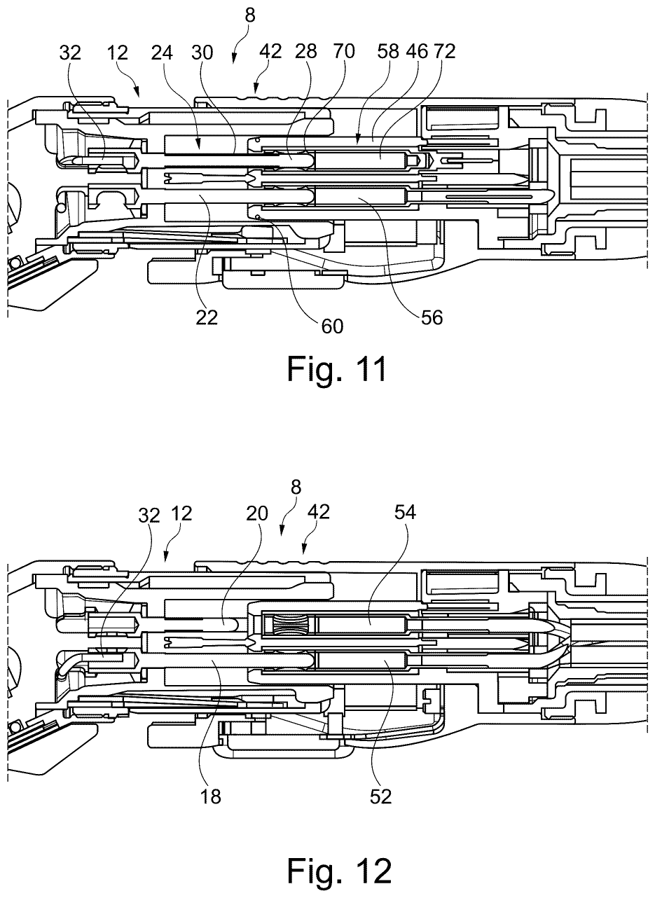

FIG. 11 illustrates a sectional view along a plane of the third and fourth coupling contacts 22, 24, 56, 58. It can be seen that only the conductive portion 28 of the fourth coupling contact 24 protrudes into the intended fourth coupling contact 58 in the coupling pin 46. The fourth coupling contact 58 in the coupling pin 46 includes a conductive portion 70 at its inlet portion and an insulating portion 72 at its bottom portion. In the OFF state, the conductive portion 28 of the fourth coupling contact 24 and the conductive portion 70 of the fourth coupling contact 58 are in engagement both mechanically and electrically.

FIG. 12 illustrates a sectional view along a plane of the first and second coupling contacts 18, 20, 52, 54. It can be seen that the second coupling contact 20 and the intended second coupling contact 54 are out of engagement in the OFF state.

Further, FIGS. 11 and 12 show the extension of the wire jumper 32 from the first coupling contact 18 to the fourth coupling contact 24 for connecting them in parallel. In the present case, the first coupling contact 18 and the fourth coupling contact 24 are arranged diagonally. However, the two coupling contacts 18, 24 can also be arranged side by side.

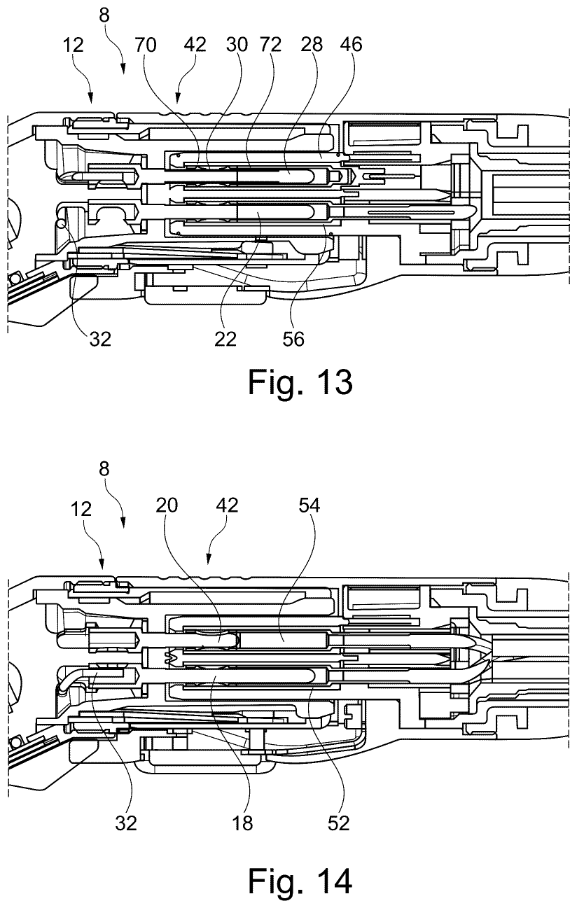

FIG. 13 illustrates a sectional view of the coupling system 8 in the ON state in the plane of the third and fourth coupling contacts 22, 24, 56, 58. It can be seen that the first coupling device 12 fully engages with the second coupling device 42 so that the coupling pin 46 is completely inserted into the coupling sleeve 16. In this configuration, the insulating portion 30 of the fourth coupling contact 24 is located opposite the conductive portion 70 of the fourth coupling contact 58, and the conductive portion 28 of the fourth coupling contact 24 is located opposite the insulating portion 72 of the fourth coupling contact 58. The fourth coupling contacts 24, 58 therefore are mechanically in engagement, but are electrically out of engagement, i.e. brought into a non-conducting state, by the above-described arrangement of the insulating portions 30, 72 and the conductive portions 28, 70. Accordingly, an electrically conductive connection between the fourth coupling contacts 24, 58 is interrupted to prevent short-circuit of the motor windings 36, 38 of the electric motor 34 (see FIG. 15).

FIG. 14 illustrates a sectional view of the coupling system 8 in the ON state in the plane of the first and second coupling contacts 18, 20, 52, 54. It can be seen that also the second coupling contact 20 and the intended second coupling contact 54 are in engagement both electrically and mechanically. As a consequence, a conductive connection is obtained between the second line 66 and the second motor winding 38. Consequently, in the ON state, all three motor windings 36, 38, 40 are electrically connected to the intended lines 64, 66, 68 of the electrical supply cable 4 in order to allow the electric motor 34 to be operated corresponding to the control by the control unit.

The afore-described configuration of the coupling contacts 18, 20, 22, 24 in the first coupling device 12 and of the coupling contacts 52, 54, 56, 58 in the second coupling device 42 can help obtain the following electric connections in the OFF state and in the ON state.

As indicated by an arrow in FIG. 16, in the OFF state a conductive connection is obtained via the first line 64, the first coupling contact 52, the first coupling contact 18, the first motor winding 36, the third motor winding 40, the third coupling contact 22, the third coupling contact 56 and the third line 68. Accordingly, the control unit may be configured to apply a measuring signal, specifically a predetermined current or a predetermined voltage, to the first line 64 and the third line 68 in order to determine a series resistance of the first motor winding 36 and the third motor winding 40. Consequently, the control unit is capable of determining a type of the electric motor 34 installed in the application part 2 based on the measured series resistance.

Moreover, as indicated by the arrow in FIG. 17, in the OFF state a conductive connection is obtained via the first line 64, the first coupling contact 52, the first coupling contact 18, the wire jumper 32, the third coupling contact 24, the third coupling contact 58, the RFID antenna 60 a shield 69 of the electrical supply cable 4. Accordingly, the RFID antenna 60 is supplied with energy to excite and read out the adjacent RFID chip 26 or to write data into the RFID chip 26. Consequently, a data transmission can be obtained between RFID chip 26 and the control unit. For example, prior to using the application part 2, data can be read out of the RFID chip 26 so that the control unit can find out whether the application part 2 is suited for the intended use. After use, the data in the RFID chip 26 can be updated. This procedure can also be performed before and after maintenance, for example.

FIG. 18 illustrates a case in which the coupling system 8 is in the ON state. Consequently, the fourth coupling contact 24 and the fourth coupling contact 58 are electrically out of engagement and the coupling contacts 18, 20, 22 of the first coupling device 12 are electrically in engagement with the intended coupling contacts 52, 54, 56 of the second coupling device 42 to allow the electric motor 34 to be operated corresponding to the control by the control unit.

In the foregoing, an embodiment of the present disclosure was described. However, the present disclosure is not limited to the above-described embodiment, and the following modification can be applied.

The tool 6 may include a further RFID chip. In this case, a further RFID antenna can be arranged adjacent to the further RFID chip in the application part 2 to read out the further RFID chip and/or to write data into the further RFID chip. The RFID antenna 60 and the further RFID antenna can be connected in series to the control unit and/or the electric circuit 62. As an alternative, the RFID antenna 60 and the further RFID antenna can be connected in parallel in separate circuits to the control unit and/or to respective electric circuits 62 for tuning the RFID antennas. When the further RFID chip is provided in the tool 6 and the RFID antenna is provided in the application part 2, data can additionally be read out/written via the tool 6 by the control unit. Consequently, transparency is further increased in the event of service or fault.

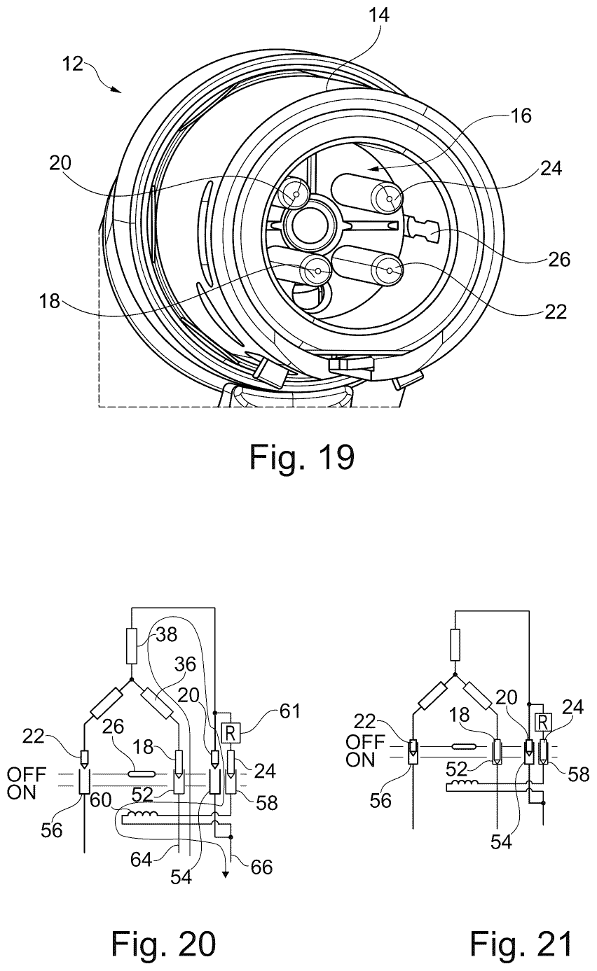

FIG. 19 illustrates a further embodiment of a first coupling device 12 in which the fourth coupling contact 24 is formed without the insulating portion 30. In the second coupling device 42, the fourth coupling contact 58 is also formed without the insulating portion 72 in this case.

As can be seen from FIG. 20, the RFID antenna 60 is arranged in the second coupling device 42. The fourth coupling contact 24 is connected in parallel to the second coupling contact 20 preferably via the wire jumper 32 in the first coupling device 12. The fourth coupling contact 58 is connected in parallel to the second coupling contact 54 in the second coupling device 42. In addition, the fourth coupling contact 24 is connected in series to a detection resistor 61. As a result, the detection resistor 61 is arranged in the application part 2 and may be encoded corresponding to the type of the application part 2 so that a type of the application part 2 can be determined by measuring a resistance between the first line 64 and the second line 66 in the OFF state. The RFID antenna 60 can be supplied with energy in the OFF state along the arrow shown in FIG. 20.

FIG. 20 further illustrates an embodiment in which the third coupling contacts 22, 56 are out of engagement electrically and mechanically in the OFF state. This configuration can also be applied to the embodiment described by way of the FIGS. 16 to 18.

It is also conceivable that the fourth coupling contacts 22, 56 that include the insulating portions 30, 72 are applied to the embodiment shown in FIG. 20. Accordingly, also the detection resistor 61 could be dispensed with.

It can be seen from FIG. 21 that, in the ON state, also the second coupling contacts 20, 54 are electrically in engagement. Due to the detection resistor 61, current from the second line 66 thus does no longer flow via the fourth coupling contacts 24, 58 but flows via the second coupling contacts 20, 54.

In the previously described embodiments, the RFID antenna 60 was always arranged in the second coupling device 42. It is also conceivable, however, that the RFID antenna 60 is arranged adjacent to the RFID chip 26 in the first coupling device 12. The RFID antenna 60 is connected in series to the further coupling contact 24. Therefore, in this case no RFID antenna 60 is positioned on the coupling pin 46.

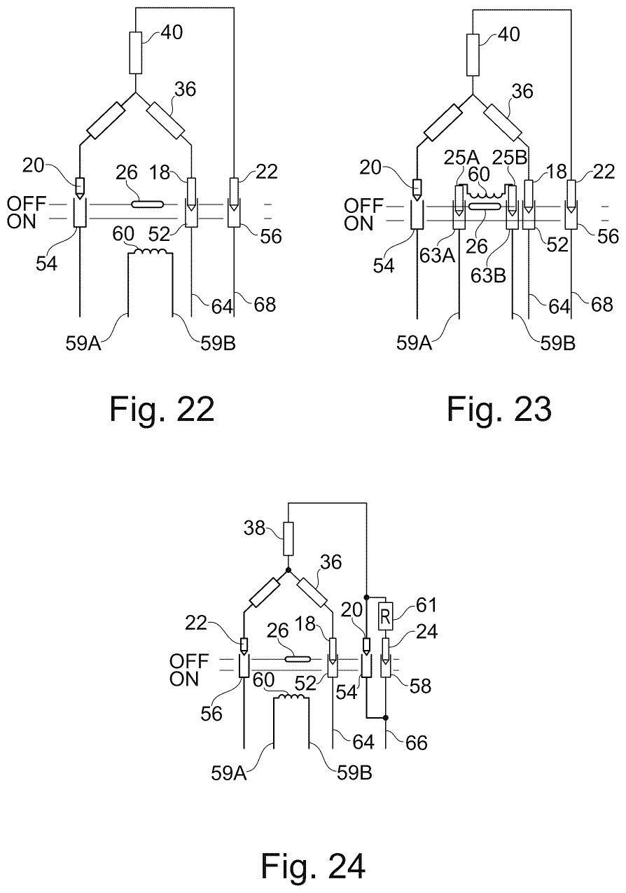

Moreover, there are conceivable further embodiments which shall be explained based on the FIGS. 22 to 24.

FIG. 22 illustrates a circuit diagram in the OFF state according to another embodiment. It can be seen that the first and second coupling devices 12, 42 only include the first to third coupling contacts 18, 20, 22, 52, 54, 56. The RFID antenna 60 is arranged in the second coupling device 42 and in the OFF state, and preferably also in the ON state, is arranged adjacent to the RFID chip 26. The RFID antenna 60 is connected and can be energized via two lines 59A and 59B which are provided in the electrical supply cable 4 in addition to the first to third lines 64, 66, 68. The two lines 59A and 59B are preferably provided by a coax cable. This type of energy supply allows the RFID chip 26 to be read out/written even when the application part is in operation. It is also conceivable, however, that one of the lines 59A or 59B is provided by the shield 69 of the electrical supply cable.

FIG. 23 illustrates a circuit diagram in the OFF state according to a further embodiment. In this embodiment, the RFID antenna 60 is arranged in the first coupling device 12. Each of the first and second coupling devices 12, 42 includes a first RFID coupling contact 25A, 63A and a second RFID coupling contact 25B, 63B which are electrically in engagement at least in the OFF state, but preferably also in the ON state. The RFID antenna 60 is connected between the first RFID coupling contact 25A and the second RFID coupling contact 25B in the first coupling device 12. In the second coupling device 42, the first RFID coupling contact 63A is connected to the line 59A and the second RFID coupling contact 63B is connected to the line 59B. The RFID coupling contacts 25A, 25B, 63A, 63B can have a smaller diameter than the coupling contacts 18, 20, 22, 52, 54, 56 which are used to supply the electric motor 34 with energy. Consequently, the RFID antenna 60 can be reliably supplied with energy preferably even when the electric motor 34 is operated.

FIG. 24 illustrates a circuit diagram in the OFF state that is similar to the circuit diagram of FIG. 22. In the first and second coupling devices 12, 42, fourth or further coupling contacts 24, 58 are provided. The fourth coupling contact 24 is connected in series to a detection resistor 61. The detection resistor 61 therefore is arranged in the application part 2 and can be encoded corresponding to the application part 2. Accordingly, a type of the application part 2 can be determined by measuring a resistance between the first line 64 and the second line 66 in the OFF state. This configuration with the fourth coupling contacts 24, 58 and the detection resistor 61 connected in series is also conceivable for the embodiment shown in FIG. 23.

### LIST OF REFERENCE SIGNS

1 surgical device
2 surgical application part
4 electrical supply cable
6 tool
8 coupling system
10 handle
12 first coupling device
14 coupling pin
16 coupling sleeve
18 first coupling contact
20 second coupling contact
22 third coupling contact
24 fourth coupling contact
25A first RFID coupling contact
25B second RFID coupling contact
26 RFID chip
28 conductive portion
30 insulating portion
32 wire jumper
34 electric motor
36 first motor winding
38 second motor winding
40 third motor winding
42 second coupling device
44 coupling sleeve
46 coupling pin
48 cable portion
50 control unit coupling device
52 first coupling contact
54 second coupling contact
56 third coupling contact
58 fourth coupling contact
59A line
59B line
60 RFID antenna
61 detection resistor
62 electric circuit
63A first RFID coupling contact
63B second RFID coupling contact
64 first line
66 second line
68 third line
69 shield
70 conductive portion
72 insulating portion

1.-30. (canceled)

31. A surgical device comprising:
a surgical application part that includes an electric motor with motor windings and a first coupling device at one end;
an electrical supply cable having a number of lines corresponding to a number of the motor windings, wherein the electrical supply cable at one end has a second coupling device arranged for coupling to the first coupling device and at a second end is connected or connectable to a control unit for controlling the electric motor, wherein the first coupling device and the second coupling device are configurable into a disconnected state or into an OFF state in which first coupling contacts of the first coupling device and the second coupling device are electrically in engagement and second coupling contacts of the first coupling device and the second coupling device are electrically out of engagement, or into an ON state in which the first coupling contacts of the first coupling device and the second coupling device and the second coupling contacts of the first coupling device and the second coupling device are electrically in engagement;
an RFID chip arranged in the first coupling device; and
an RFID antenna arranged in the first coupling device and adapted to be supplied with energy which is arranged adjacent to the RFID chip and is configured to excite the RFID chip in order to transmit data between the RFID chip and the control unit,
wherein each of the first coupling device and the second coupling device comprises a third coupling contact, the third coupling contacts being electrically in engagement at least in the OFF state in order to supply the RFID antenna with energy.

32. The surgical device according to claim 31, wherein the first coupling device and the second coupling device include third coupling contacts which are electrically in engagement in the OFF state and in the ON state.

33. The surgical device according to claim 31, wherein the first coupling device and the second coupling device include third coupling contacts which are electrically out of engagement in the OFF state and are electrically in engagement in the ON state.

34. The surgical device according to claim 31, wherein the third coupling contact in the first coupling device is connected in parallel to the second coupling contact of the first coupling device via the RFID antenna.

35. The surgical device according to claim 31, wherein the third coupling contact in the first coupling device is connected in parallel to the first coupling contact of the first coupling device via the RFID antenna.

36. The surgical device according to claim 34, wherein the third coupling contact in the second coupling device is connected in parallel to the second coupling contact of the second coupling device.

37. The surgical device according to claim 34, wherein the third coupling contact in the second coupling device is connected to a shield of the electrical supply cable.

38. The surgical device according to claim 34, wherein the third coupling contacts are configured so that they are electrically in engagement also in the ON state, and one of the third coupling contacts is connected in series to a detection resistor.

39. The surgical device according to claim 34, wherein the third coupling contacts are configured so that they are electrically out of engagement in the ON state.

40. The surgical device according to claim 39, wherein one of the third coupling contacts is connected in series to a detection resistor.

41. The surgical device according to claim 39, wherein:
the first coupling contact, the second coupling contact and the third coupling contact in the first coupling device comprise pins and the first coupling contact, the second coupling contact and the third coupling contact in the second coupling device comprise bushes,
in the first coupling device, the third coupling contact is longer than the second coupling contact, and
the third coupling contacts include insulating portions that are configured so that the third coupling contacts are electrically out of engagement in the ON state.

42. A surgical device, comprising:
a surgical application part that includes an electric motor with motor windings and a first coupling device at one end;
an electrical supply cable having a number of lines corresponding to a number of the motor windings, wherein the electrical supply cable at a second end includes a second coupling device arranged for coupling to the first coupling device and at the second end is connected or connectable to a control unit for controlling the electric motor;
wherein the first coupling device and the second coupling device are arranged so that they can be brought into a disconnected state or into an OFF state in which first coupling contacts of the first coupling device and the second coupling device are electrically in engagement and second coupling contacts of the first coupling device and the second coupling device are electrically out of engagement, or into an ON state in which the first coupling contacts of the first coupling device and the second coupling device and the second coupling contacts of the first coupling device and the second coupling device are electrically in engagement;
an RFID chip arranged in the first coupling device; and
an RFID antenna arranged in the second coupling device and adapted to be supplied with energy which, at least in the OFF state, is arranged adjacent to the RFID chip and is configured to excite the RFID chip to transmit data between the RFID chip and the control unit,
wherein each of the first coupling device and the second coupling device includes a third coupling contact which are arranged so that they are electrically in engagement at least in the OFF state in order supply the RFID antenna with energy.

43. The surgical device according to claim 42, wherein the first coupling device and the second coupling device include third coupling contacts which are electrically in engagement in the OFF state and in the ON state.

44. The surgical device according to claim 42, wherein the first coupling device and the second coupling device include third coupling contacts which are electrically out of engagement in the OFF state and are electrically in engagement in the ON state.

45. The surgical device according to claim 42, wherein the third coupling contact in the first coupling device is connected in parallel to the second coupling contact of the first coupling device.

46. The surgical device according to claim 42, wherein the third coupling contact in the first coupling device is connected in parallel to the first coupling contact of the first coupling device.

47. The surgical device according to claim 45, wherein the third coupling contact in the second coupling device is connected in parallel to the second coupling contact of the second coupling device via the RFID antenna.

48. The surgical device according to claim 45, wherein the third coupling contact in the second coupling device is connected to a shield of the electrical supply cable via the RFID antenna.

49. The surgical device according to claim 45, wherein the third coupling contacts are electrically in engagement even in the ON state, and one of the third coupling contacts is connected in series to a detection resistor.

50. The surgical device according to claim 45, wherein the third coupling contacts are electrically out of engagement in the ON state.

51. The surgical device according to claim 50, wherein one of the third coupling contacts is connected in series to a detection resistor.

52. The surgical device according to claim 50, wherein:
the first coupling contact, the second coupling contact and the third coupling contact in the first coupling device comprise pins, and the first coupling contact, the second coupling contact and the third coupling contact in the second coupling device comprise bushes,
in the first coupling device the third coupling contact is longer than the second coupling contact, and
the third coupling contacts of the first coupling device and the second coupling device include insulating portions which are configured so that the third coupling contacts of the first coupling device and the second coupling device are electrically out of engagement in the ON state.

53. A surgical device, comprising:
a surgical application part which includes an electric motor with motor windings and a first coupling device at one end;
an electrical supply cable having a number of lines corresponding to a number of the motor windings, wherein the electrical supply cable at one end includes a second coupling device arranged for coupling to the first coupling device and at a second end is connected or connectable to a control unit for controlling the electric motor, wherein the first coupling device and the second coupling device are arranged so that they can be brought into a disconnected state or into an OFF state in which first coupling contacts of the first coupling device and the second coupling device are electrically in engagement and second coupling contacts of the first coupling device and the second coupling device are electrically out of engagement, or into an ON state in which the first coupling contacts of the first coupling device and the second coupling device and the second coupling contacts of the first coupling device and the second coupling device are electrically in engagement,
an RFID chip disposed in the first coupling device; and
an RFID antenna disposed in the second coupling device and adapted to be supplied with energy which at least in the OFF state is arranged adjacent to the RFID chip and is configured to excite the RFID chip in order to transmit data between the RFID chip and the control unit,
wherein two further lines are provided in the electrical supply cable and connected to the RFID antenna in the second coupling device to supply the RFID antenna with energy.

54. The surgical device according to claim 53, wherein one of the two further lines is provided by a shield of the electrical supply cable.

55. The surgical device according to claim 53, wherein the two further lines are provided by a coax cable.

56. The surgical device according to claim 53, wherein:
the first coupling device and the second coupling device comprise third coupling contacts connected in parallel to the second coupling contacts of the first coupling device and the second coupling device, the third coupling contacts being electrically in engagement at least in the OFF state, and
one of the third coupling contacts is connected in series to a detection resistor.

57. A surgical device comprising:
a surgical application part which includes an electric motor with motor windings and a first coupling device at one end;
an electrical supply cable having a number of lines corresponding to a number of the motor windings, wherein the electrical supply cable at one end includes a second coupling device arranged for coupling to the first coupling device, and at a second end is connected or connectable to a control unit for controlling the electric motor, wherein the first coupling device and the second coupling device are arranged so that they can be brought into a disconnected state or into an OFF state in which first coupling contacts of the first coupling device and the second coupling device are electrically in engagement and second coupling contacts of the first coupling device and the second coupling device are electrically out of engagement, or into an ON state in which the first coupling contacts of the first coupling device and the second coupling device and the second coupling contacts of the first coupling device and the second coupling device are electrically in engagement;
an RFID chip disposed in the first coupling device; and
an RFID antenna disposed in the first coupling device and adapted to be supplied with energy which is arranged adjacent to the RFID chip and is configured to excite the RFID chip in order to transmit data between the RFID chip and the control unit,
wherein:
each of the first coupling device and the second coupling device includes two RFID coupling contacts which are electrically in engagement at least in the OFF state,
the two RFID coupling contacts in the first coupling device are connected to the RFID antenna, and
the two RFID coupling contacts in the second coupling device are connected to two further lines which are provided in the electrical supply cable for supplying the RFID antenna with energy.

58. The surgical device according to claim 57, wherein one of the two further lines is provided by a shield of the electrical supply cable.

59. The surgical device according to claim 57, wherein the two further lines are provided by a coax cable.

60. The surgical device according to claim 57, wherein:
the first coupling device and the second coupling device further comprise third coupling contacts that are electrically in engagement at least in the OFF state, and at least one of the third coupling contacts is connected in series to a detection resistor.
